(19) United States
(12) Patent Application Publication
(10) Pub. No.: US 2015/0196232 A1
(43) Pub. Date: Jul. 16, 2015

(54) SYSTEM AND METHOD FOR TESTING MOTOR AND COGNITIVE PERFORMANCE OF A HUMAN SUBJECT WITH A MOBILE DEVICE

(71) Applicant: Apptomics LLC, Wellesley Hills, MA (US)

(72) Inventors: Georgia Mitsi, Wellesley Hills, MA (US); Spyridon Papapetropoulos, Wellesley Hill, MA (US)

(73) Assignee: Apptromics LLC

(21) Appl. No.: 14/594,766

(22) Filed: Jan. 12, 2015

Related U.S. Application Data
(60) Provisional application No. 61/927,722, filed on Jan. 15, 2014.

(51) Int. Cl. A61B 5/11; G09B 19/00; A61B 5/16; A61B 5/00

(52) U.S. Cl. CPC A61B 5/1124; A61B 5/6898; G09B 19/00; A61B 5/162

(57) ABSTRACT

A system for testing motor and/or performance of a human subject with a mobile device is provided. The system features a mobile device including a processor subsystem and configured to receive taps from a hand or part of a hand of a human subject on a screen of the mobile device. A tap application for the mobile device is executed by the processor subsystem and is responsive to taps by the hand or part of the hand on the screen and is configured to measure the frequency and duration of each tap by the hand or part of the hand on the screen to determine the motor and/or cognitive performance of the human subject.

### RELATED APPLICATIONS

This application claims benefit of and priority to U.S. Provisional Application Ser. No. 61/927,722, filed Jan. 15, 2014 under 35 U.S.C. §§119, 120, 363, 365, and 37 C.F.R. §1.55 and §1.78 which is incorporated herein by this reference.

### FIELD OF THE INVENTION

This invention relates to a system and method for testing motor and cognitive performance of a human subject with a mobile device.

### BACKGROUND OF THE INVENTION

Disease and normal aging can influence motor task execution, e.g., hand or finger movement. The conventional finger tapping test and hand pronation-supination test are basic tests that may be used by health care professionals for evaluating movement patterns. Both are commonly used in routine clinical examination as part of clinical research protocols and neurophysiological examinations. See, e.g., Stavrinou et al., Evaluation of cortical connectivity during real and imagined rhythmic finger tapping, Brain topography 19, 137 (Spring 207), and Leijnse et al., Assessment of individual finger muscle activity in the extensor digitorum communis by surface EMG, Journal of neurophysiology 100, 3225 (December 2008), both incorporated by reference herein. Test results are typically interpreted subjectively based on the experience of the healthcare professional or the researcher. Such conventional tests allow for drawing conclusions on several key elements that may be subsequently used as complementary data for characterizing disease profiles, such as arrythmokinesis, which may include but is not limited to, hastening, faltering, or freezing in a tapping pattern, hypokinesia in Parkinson's disease, and the like. See, e.g., Arnold et al., Sensitivity and specificity of finger tapping test scores for the detection of suspect effort, The Clinical neuropsychologist 19, 105 (February 2005), and Nakamura et al., Disturbances of rhythm formation in patients with Parkinson's disease: Part I. Characteristics of tapping response to the periodic signals, Perceptual and motor skills 46, 53 (February 1978), both incorporated by reference herein.

Despite their conceptual simplicity, the conventional finger tapping and hand pronation-supination tests provide highly valuable information and are considered sensitive markers for the detection of alteration and rhythm formation due to aging, conditions such as cognitive decline/dementia, drug abuse and dependency, trauma, metal poisoning, developmental, neurological, autoimmune, musculoskeletal and psychiatric diseases. See, e.g., Shimoyama et al., The finger-tapping test. A quantitative analysis, Archives of neurology 47, 681 (June 1990), Dean et al., Dementia and effort test performance, The Clinical neuropsychologist 23, 133 (January 2009), Flavel et al., Abnormal maximal finger tapping in abstinent cannabis users, Human psychopharmacology, (Sep. 23, 2013), and Blond et al., Neuromotor function in a cohort of Danish steel workers, Neurotoxicology 28, 336 (March 2007); all incorporated by reference herein.

Despite their utility, several factors can influence test results and interpretation. Tools to objectively capture and quantify hand and finger movements would help with the diagnosis and management of at least the aforementioned diseases and conditions. Additionally, there is evidence that repetition or practice of such tests may enhance motor performance indicating the therapeutic potential of cognitive gaming patterns used in simple tapping approaches. See, e.g., Koeneke et al., How finger tapping practice enhances efficiency of motor control, Neuroreport 17, 1565 (Oct. 23, 2006), incorporated by reference herein.

Conventional systems and methods to test the motor and/or cognitive performance of a human subject typically do not include the duration and accuracy of taps by the hand or part of the hand on the screen or a target on the screen when determining motor and/or cognitive performance of a human subject. This may result in inaccurate, ineffective and inefficient results. Thus, there is a need for a system and method which can accurately, effectively, and efficiently test the motor and cognitive performance of a human subject.

### BRIEF SUMMARY OF THE INVENTION

In one aspect, a system for testing motor and/or performance of a human subject with a mobile device is featured. The system includes a mobile device including a processor subsystem and configured to receive taps from a hand or part of a hand of a human subject on a screen of the mobile device. A tap application for the mobile device is executed by the processor subsystem and responsive to taps by the hand or part of the hand on the screen and configured to measure the frequency and duration of each tap by the hand or part of the hand on the screen to determine the motor and/or cognitive performance of the human subject.

In one embodiment, the tap application may be configured to calculate the duration of each tap by determining the total amount of time the hand or part of the hand touches the screen per tap during a predetermined amount of time. The taps by the hand or part of the hand may include tapping on the screen with a finger or thumb of the hand. The taps by the hand or part of the hand on the screen may include alternatively tapping on the screen with the palm and dorsal surface of the hand in a repetitive pronation-supination movement. The tap application may be configured to display a target of a predetermined shape having a center point on the screen. The tap application may be configured to determine the accuracy of the taps on or near the center point of the target to further determine the motor performance of the human subject. The tapping application may be configured to determine the accuracy of the taps on or near the center point by measuring the distance from the location of each tap on the screen and the center point of the target. The tap application may be configured to display a plurality of spaced targets of a predetermined shape and each including a center point on the screen, the tapping application configured to measure the frequency and duration of taps inside or on each of the spaced target by the hand or part of the hand to determine the motor performance of the human subject. The tap application may be configured to calculate the average duration of each tap by determining the total amount of time the hand or part of the hand touches each of the spaced target per tap during a predetermined amount of time. The tapping application may be configured to measure the accuracy of the taps inside or on the spaced targets to determine the motor performance of the human subject. The tapping application may be configured to determine the accuracy by measuring the distance between the location of each tap on the screen and a center point of an intended spaced target. The predetermined pattern may include a complex pattern of taps inside or on the spaced targets to measure cognitive performance of the human subject. The tapping application may be configured to display a plurality of spaced targets of a predetermined shape with a center point on the screen, the tapping application configured to measure the frequency of taps inside or on each of the spaced targets to determine the motor performance of the human subject. The tapping application may be configured to measure tapping velocity of alternating taps between the spaced targets in a predetermined pattern to determine the motor performance of the human subject. The predetermined pattern may include alternately tapping inside or on the spaced targets. The predetermined shape may include circular shape having one or more concentric rings about the center point. The mobile device may include a mobile smart phone, a tablet device, a laptop computer, a portable personal computer, or a notebook. The tap application may be configured to generate one or more reports providing an indication of the motor and/or cognitive performance of the human subject. The one or more reports may include one or more of one a target test report, a pronation-supination report, and a two target test report.

In another aspect, a system for testing the motor and/or cognitive performance of a human subject with a mobile device is featured. The system includes a mobile device including a processor subsystem screen configured to receive taps from a hand or part of a hand of a human subject on a screen of the mobile device. A tapping application for the mobile device is executed by the processor subsystem and configured to display a plurality of spaced targets of a predetermined shape on the screen and configured to measure the frequency, duration, and accuracy of taps inside or on the spaced targets by the hand or part of the hand in a predetermined pattern to determine the motor and/or cognitive performance of the human subject.

In another aspect, a system for testing the motor and cognitive performance of a human subject is featured. The system includes a mobile device including a processor subsystem and configured to receive taps by a hand or part of the hand of a human subject on a screen of the mobile device. A tap application for the mobile device executed by the processor subsystem configured to display a plurality of targets in a predetermined complex static or moving pattern on the screen and configured to measure the accuracy and duration of correct taps inside or on each of the plurality of spaced targets and the tapping velocity of taps between the spaced targets to determine the motor and cognitive performance of the human subject.

In another aspect, a method for testing the motor and/or cognitive performance of a human subject with a mobile device is featured. The method includes receiving taps from a hand or part of a hand of a human subject on a screen of the mobile device and measuring the frequency and duration of each tap by the hand or part of the hand on the screen to determine the motor performance of the human subject.

In one embodiment, the method may include calculating the duration of each tap by determining the total amount of time the hand or part of the hand touches the screen per tap during a predetermine amount of time. The method may include the step of displaying a target of a predetermined shape and having a center point on the screen. The method may include determining the accuracy of taps on or near the center point of the target to further determine the motor performance of the human subject. The accuracy may include measuring the distance from the location of each tap on the screen and the center point of the target. The method may further include displaying a plurality of spaced targets of a predetermined shape and each having a center point on the screen and measuring the frequency and duration of taps inside or on each of the spaced targets to determine the motor performance of the human subject. The method may further include measuring the accuracy of taps inside or on the spaced targets to determine the motor performance of the human subject. The method may further include measuring the tapping velocity of alternating taps between the spaced targets in a predetermined pattern to determine motor performance of the human subject. The predetermined pattern may include alternatingly tapping inside or on the spaced targets.

In another aspect, a method for testing the motor and/or cognitive performance of a human subject with a mobile device is featured. The method includes receiving taps from a hand or part of a hand of a human subject on a screen of the mobile device, and displaying a plurality of spaced targets of a predetermined shape on the screen and measuring the frequency, duration, and accuracy of taps inside or on the spaced targets by the hand or part of the hand in a predetermined pattern to determine the motor and/or cognitive performance of a human subject.

In yet another aspect, a method for testing the motor and/or cognitive performance of human subject with a mobile device is featured. The method includes receiving taps by hand or part of the hand of a human subject on a screen of a mobile device, and displaying a plurality of targets in a predetermined complex static or moving pattern on the screen and measuring the accuracy and duration of correct taps inside or on each of the plurality of spaced targets and determining the tapping velocity of taps between the spaced targets to determine the motor and cognitive performance of a human subject.

The subject invention, however, in other embodiments, need not achieve all these objectives and the claims hereof should not be limited to structures or methods capable of achieving these objectives.

### BRIEF DESCRIPTION OF THE SEVERAL VIEWS OF THE DRAWINGS

Other objects, features and advantages will occur to those skilled in the art from the following description of a preferred embodiment and the accompanying drawings, in which:

FIG. 1 is a schematic diagram showing one embodiment of the system and method for testing the motor and/or cognitive performance of a human subject with a mobile device of this invention;

FIG. 2 is a schematic diagram of another embodiment of the system and method for testing the motor and/or cognitive performance of a human subject with a mobile device;

FIG. 3 is a flow chart depicting one example of the primary steps associated with calculating the frequency of the taps on the screen for the system shown in FIGS. 1 and 2;

FIG. 4 is a flow chart showing one example of the primary steps associated with calculating the duration of taps on the screen for the system shown in FIGS. 1 and 2;

FIG. 5 is a flow chart depicting one example of the primary steps associated with calculating the frequency of taps inside the target shown in FIG. 1;

FIG. 6 is a flow chart depicting one example of the primary steps associated with calculating each tap duration of taps in the target shown in FIG. 1;

FIG. 7 is a flow chart depicting one example of the primary steps associated with determining the accuracy of taps inside the target shown in FIG. 1;

FIG. 8 is a schematic diagram of one embodiment of the system and method for testing the motor and/or cognitive performance of a human subject with a mobile device in accordance with the subject invention;

FIG. 9 is a flow chart depicting one example of the primary steps associated with determining the frequency of taps inside the spaced targets shown in FIG. 8;

FIG. 10 is a flow chart depicting one example of the primary steps associated with determining the tap duration on each of the spaced targets shown in FIG. 8;

FIG. 11 is a flow chart depicting one example of the primary steps associated with determining the accuracy of taps on the spaced targets shown in FIG. 8;

FIG. 12 is a flow chart depicting one example of the primary steps associated with determining the velocity of taps between the spaced targets shown in FIG. 8; and

FIGS. 13-15 show examples of various reports that may be generated by the system shown in one or more of FIGS. 1-12.

### DETAILED DESCRIPTION OF THE INVENTION

Aside from the preferred embodiment or embodiments disclosed below, this invention is capable of other embodiments and of being practiced or being carried out in various ways. Thus, it is to be understood that the invention is not limited in its application to the details of construction and the arrangements of components set forth in the following description or illustrated in the drawings. If only one embodiment is described herein, the claims hereof are not to be limited to that embodiment. Moreover, the claims hereof are not to be read restrictively unless there is clear and convincing evidence manifesting a certain exclusion, restriction, or disclaimer.

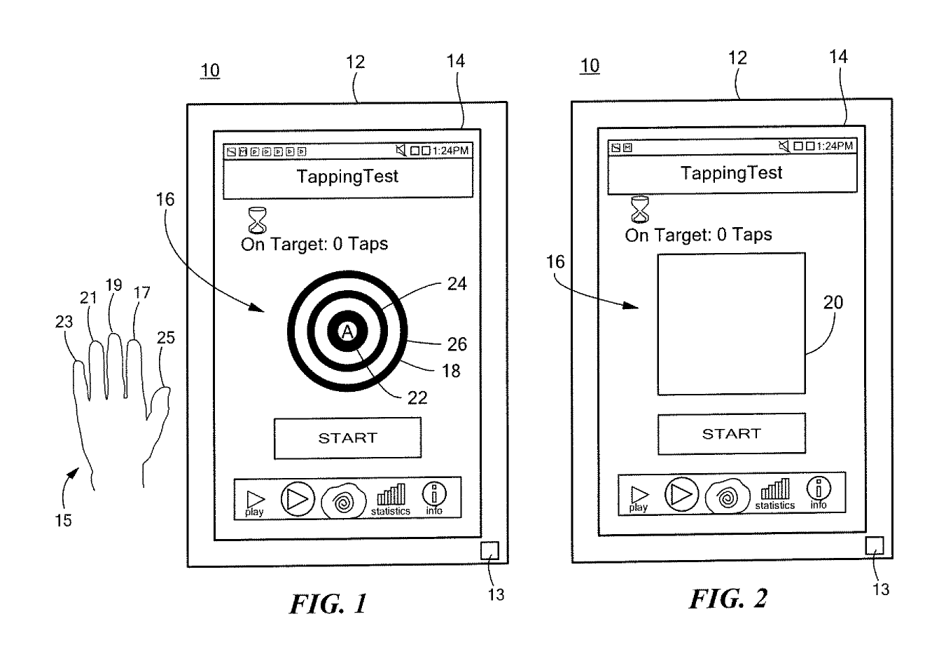

There is shown in FIG. 1 one embodiment of system 10 for testing the motor and/or cognitive performance of a human subject for a mobile device. System 10 includes mobile device 12 including processor subsystem 13 and screen 14 configured to receive taps from a human subject thereon. Mobile device 12 may include a computing device that includes processor 13 for executing a software application, as known by those skilled in the art. For example, mobile device 12 may include smart phones, tablets, laptop computers, portable personal computers, notebooks, wearable devices with screens, and/or other mobile devices. Various application platforms exist for different operating systems may be used on mobile device 12, such as Microsoft Windows® platforms, Google Android® platforms, and Apple Mac OS and iOS® platforms.

System 10 also includes tap application 16 for mobile device 12 executed by the processor subsystem 13 and responsive to taps by hand 15 or part of a hand 15 of a human subject on screen 14. An application, also referred to as an “app,” generally refers to a software application that executes on a computing device, such as a mobile device 12.

Tap application 16 is configured to measure the frequency, or the number of taps by hand 15 o part of hand 15 on screen 14 in a predetermined amount of time, e.g., taps per second, and the duration of each tap by hand 15 or part of hand 15 on the screen to determine the motor performance of a human subject.

In one example, tap application 16 may be configured to calculate the duration of each tap on screen 14 by determining the total amount of time hand 15 or part of hand 15 touches screen 14 per tap during a predetermined amount of time and dividing the total amount of time by a total number of taps during the predetermined amount of time. The tap duration may be the average amount of time hand 15 or part of hand 15, touches screen 14 per tap during 30 seconds. Thus, the tap duration is calculated as the time difference between when hand 15 or part of hand 15 touches screen 14 and when hand or part of hand 15 leaves screen 14 per tap.

In one example, the taps on screen 14 by hand 15 or part of hand 15 may include tapping screen 14 with finger or thumb of a human subject, e.g., index finger 17, or any of fingers 19, 21, or 23 or thumb 25. In the example shown in FIG. 1, the user taps on target 18 and tap application 16 determines the frequency, or total number of taps on target 18 in a predetermined amount of time, and the duration of each tap on target 18 to determine the motor performance of the human subject. The predetermined amount of time can be any desired amount of time specified by a healthcare professional, e.g., is about 30, 60, or 90 seconds.

In another example, the human subject may tap on screen 14, FIG. 2, in an alternating manner with the palm and dorsal surface of hand 15 of the human subject in a repetitive pronation-supination movement on area 20 or screen 14. Tap application 16 is configured to determine the total number of taps on area 20 by the palm or dorsal surface in a predetermined amount of time and the duration of each tap in area 20 to determine the motor performance of the human subject.

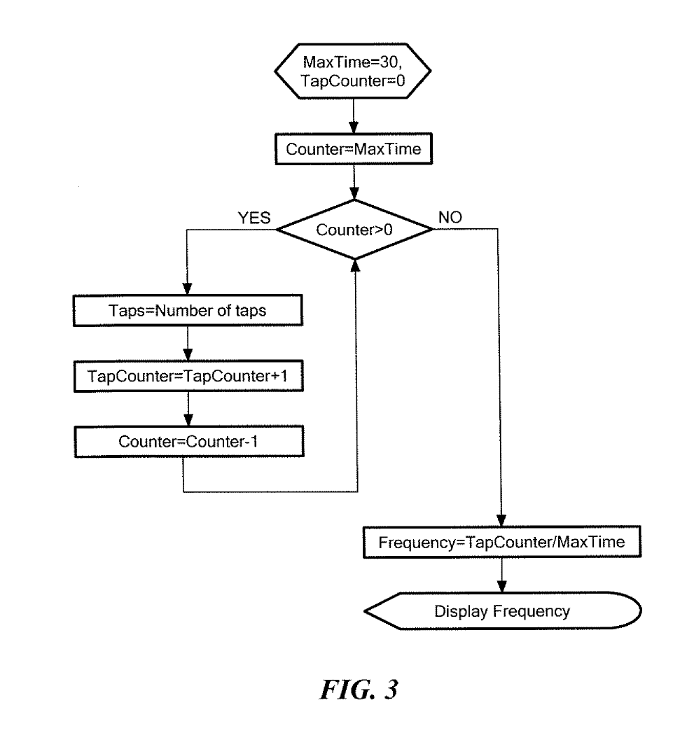

FIG. 3 shows one example of a flow chart depicting the primary steps associated with one example of calculating the frequency of taps on screen 14, FIGS. 1 and 2, by tap application 16.

For enablement purposes only, the following code portion is provided which can be executed by the processor subsystem on mobile device 12 to carry out the primary steps and/or functions of the application 16 discussed above and recited in the claims hereof. Other equivalent algorithms and code can be designed by a software engineer and/or programmer skilled in the art using the information provided therein:

Frequency

```
Steps:
  1. Set MaxTime=30
  2. Set TapCounter=0
  3. Set Counter=MaxTime
  4. While Counter>0 Do
    1. Set Taps=number of taps
    2. Set TapCounter=TapCounter+Taps
    3. Set Counter=Counter−1
  5. EndWhile
  6. Set Frequency=TapCounter/MaxTime
  7. Output Frequency
```

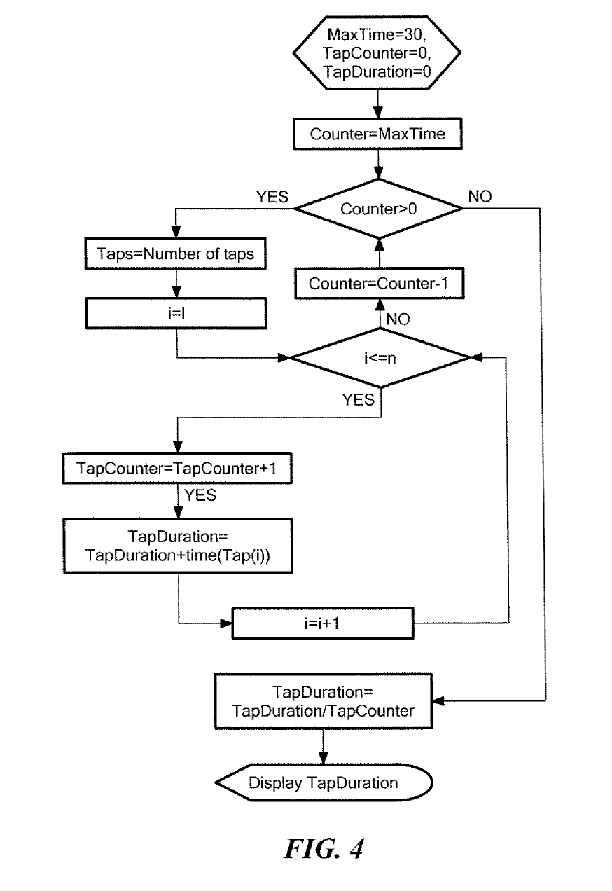

One example of a flow chart showing the primary steps associated with calculating the duration of the taps on screen 14, FIGS. 1 and 2 by tap application 16 is shown in FIG. 4.

For enablement purposes only, the following code portion is provided which can be executed by the processor subsystem on mobile device 12 to carry out the primary steps and/or functions of the application 16 discussed above and recited in the claims hereof. Other equivalent algorithms and codes can be designed by a software engineer and/or programmer skilled in the art using the information provided herein.

Duration

```
Steps:
  1. Set MaxTime=30
  2. Set TapCounter=0
  3. Set TapDuration=0
  4. Set Counter=MaxTime
  5. While Counter>0 Do
    1. Set Taps=number of taps
    2. For i=1 to Taps do
      1. Set TapCounter=TapCounter+1
      2. Set TapDuration=TapDuration+Time(Tapi )
    3. EndFor
    4. Set Counter=Counter−1
  6. EndWhile
  7. Set TapDuration=TapDuration/TapCounter
  8. OutputTapDuration
Where Time( ) is a function measuring the duration of a Tap in miliseconds.
```

In one embodiment, tap application 16, FIG. 1, is configured to provide target 18 having a predetermined shape, e.g., a circular shape as shown having center point 22, or any type shape with a center point. Preferably, target 18 may also include one or more concentric rings, e.g., concentric rings 24 and 26.

In this example, tap application 16 is configured to measure the frequency of taps by the human subject inside or on target 18 and the duration of each of the taps on target 18 to determine the motor performance of the human subject. The duration is preferably calculated as discussed above.

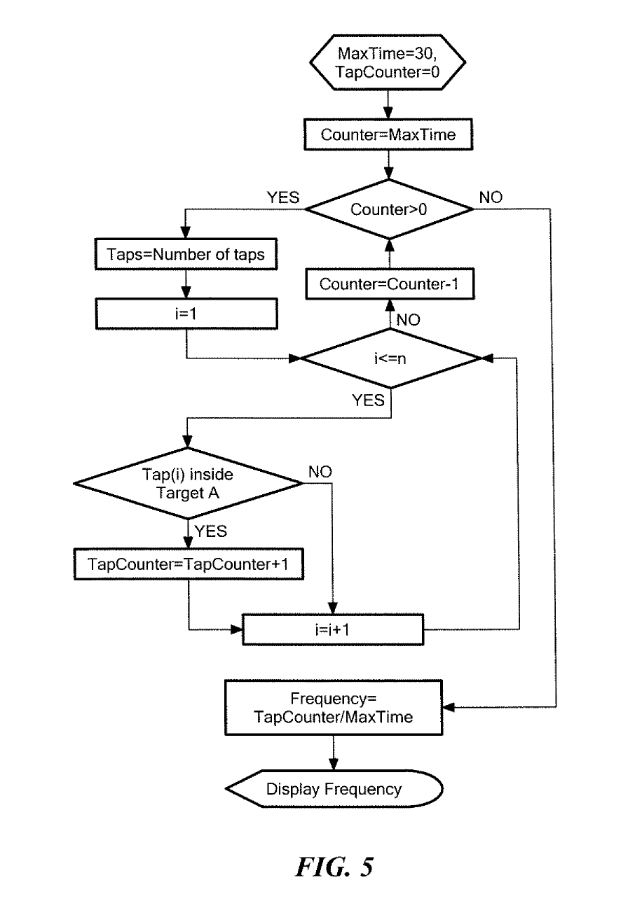

FIG. 5 is a flow chart depicting the primary steps associated with one example calculating the frequency of taps inside target 18 performed by tap application 16.

For enablement purposes only, the following code portion is provided which can be executed by the processor subsystem on mobile device 12 to carry out the primary steps and/or functions of the application 16 discussed above and recited in the claims hereof. Other equivalent algorithms and codes can be designed by a software engineer and/or programmer skilled in the art using the information provided herein.

One Target Test
Frequency

```
Steps:
  1. Set MaxTime=30
  2. Set TapCounter=0
  3. Set Counter=MaxTime
  4. While Counter>0 Do
    4.1. Set Taps = number of Taps
    4.2. For i=1 to Taps do
      a) If (Tapi inside target A) Then
        Set TapCounter=TapCounter+1
        b) EndIf
    4.3. EndFor
  5. Set Counter=Counter−1
  6. EndWhile
  7. Set Frequency=TapCounter/MaxTime
  8. Output Frequency
```

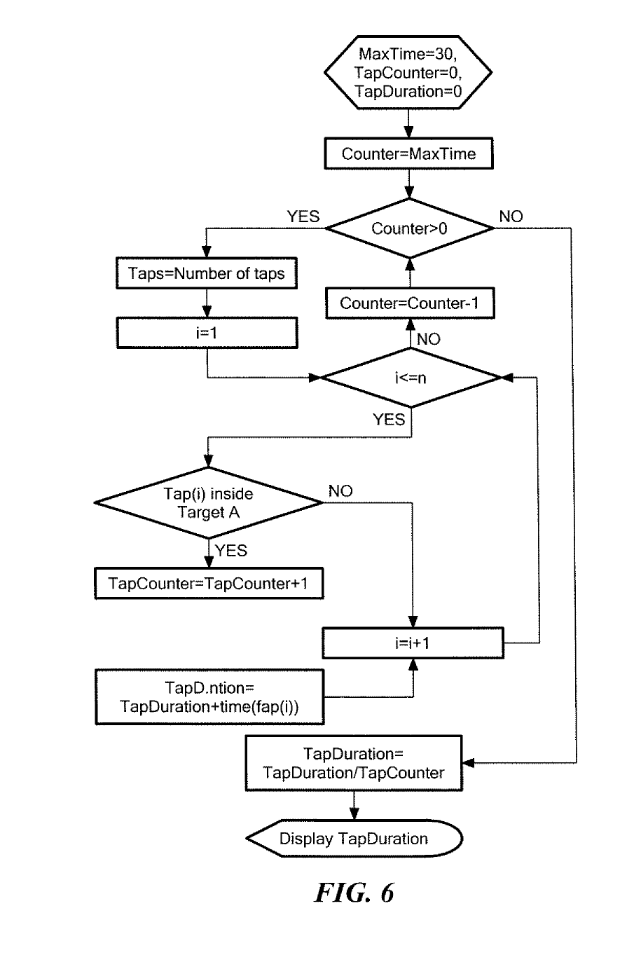

FIG. 6 is a flow chart showing one example of the primary steps associated with determining the duration of each tap by the human subject inside or on target 18, FIG. 1 performed by tap application 16.

For enablement purposes only, the following code portion is provided which can be executed by the processor subsystem on mobile device 12 to carry out the primary steps and/or functions of the application 16 discussed above and recited in the claims hereof. Other equivalent algorithms and code can be designed by a software engineer and/or programmer skilled in the art using the information provided herein.

Duration

```
Steps:
    7. Set MaxTime=30
    8. Set TapCounter=0
    9. Set Counter=MaxTime
  10. Set TapDuration=0
  11. While Counter>0 Do
    1. Set Taps=number of taps
    2. For i=1 to Taps do
      1. If (Tapi inside target A) Then
        1. Set TapCounter=TapCounter+1
        2. Set TapDuration=TapDuration+Time(Tapi )
          2. EndIf
    3. EndFor
  12. Set Counter=Counter−1
    7. End While
    8. Set Duration=Duration/TapCounter
    9. Output Duration
Where Time( ) is a function measuring the duration of a Tap in miliseconds.
```

Tap application 16 may also determine the accuracy of taps on or near center point 22 of target 18 to further determine the motor performance of the human subject. Preferably, tap application 16 is configured to determine the accuracy of the taps on target 18 by measuring the distance between the location of each tap on the screen and center point 22 of target 18.

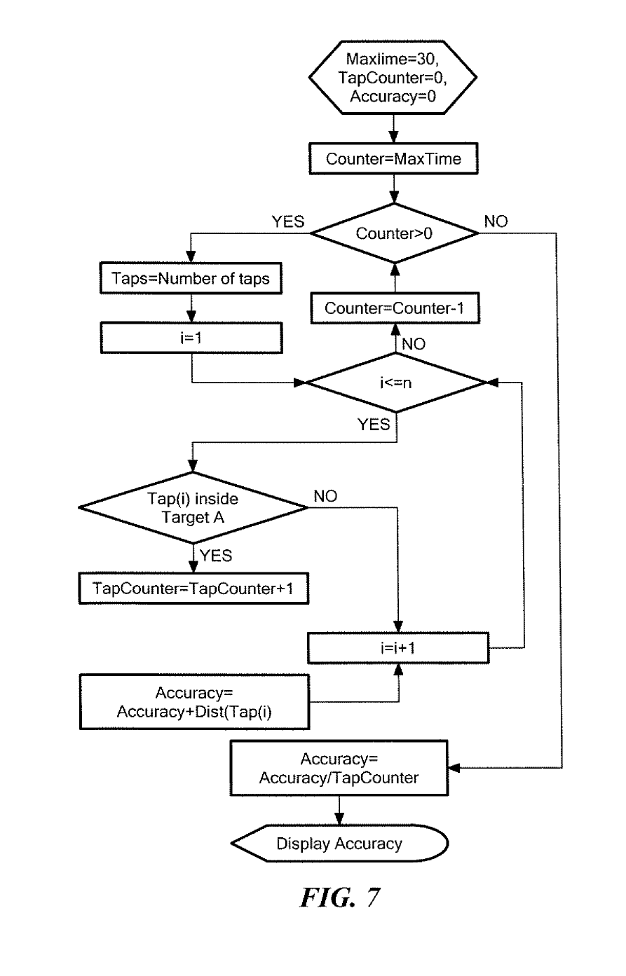

FIG. 7 is a flow chart depicting the primary steps associated of one example of calculating the accuracy of the taps by the human subject on target 18, FIG. 1 performed by tap application 16.

For enablement purposes only, the following code portion is provided which can be executed by the processor subsystem on mobile device 12 to carry out the primary steps and/or functions of the application 16 discussed above and recited in the claims hereof. Other equivalent algorithms and code can be designed by a software engineer and/or programmer skilled in the art using the information provided herein.

Accuracy:

```
1. Set MaxTime=30
2. Set TapCounter=0
3. Set Accuracy=0
4. Set Counter=MaxTime
5. While Counter>0 Do
  1. Set Taps=number of taps
  2. For i=1 to Taps do
    1. If (Tapi inside target A) Then
      1. Set TapCounter=TapCounter+1
      2. Set Accuracy=Accuracy +Dist(Tapi,TargetA)
        2. EndIf
  3. EndFor
6. Set Counter=Counter−1
7. EndWhile
8. Set Accuracy = Accuracy/TapCounter
9. Output Accuracy
Where Dist(X,Target) is a function which measures the distance from the center of Target.
```

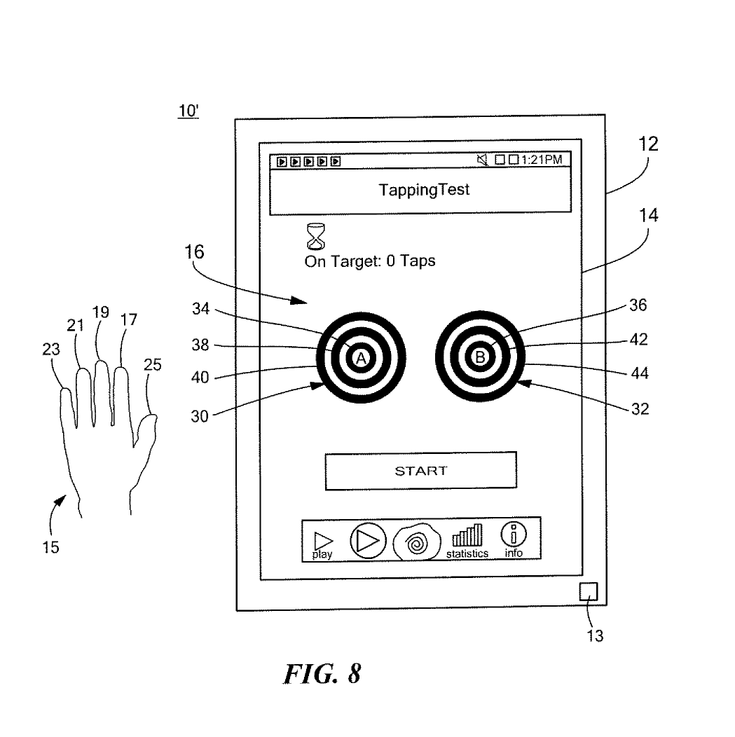

In another embodiment, system 10′, FIG. 8, where like parts have been given like numbers, includes tap application 16 which includes a plurality of spaced targets 30, 32, similar to target 18, FIG. 1, which each include center points 34, 36, FIG. 8, and concentric rings 38, 40, and 42, 44, respectively. In one example, tap application 16 is configured to measure the frequency and duration of alternating taps inside target 30 and 32 to determine the motor performance of the human subject.

In this example, tap application 16 is configured to calculate the average duration of each tap inside or on each of spaced targets 30, 32 by determining the total amount of time hand or part of hand 15 touches each of spaced targets 30, 32 per tap during a predetermined amount of time and dividing the total amount of time by the total number of taps during the predetermined amount of time, similar as discussed above with reference to FIG. 1.

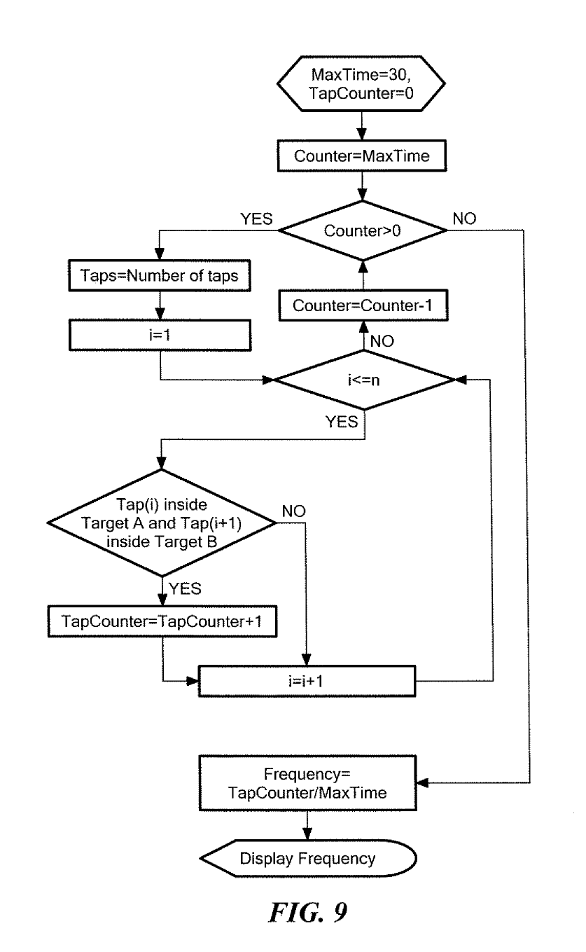

FIG. 9 is a flow chart depicting the primary steps associated with one example of calculating the frequency of taps inside targets 30 and 32 performed by tape application 16.

For enablement purposes only, the following code portion is provided which can be executed by the processor subsystem on mobile device 12 to carry out the primary steps and/or functions of the application 16 discussed above and recited in the claims hereof. Other equivalent algorithms and code can be designed by a software engineer and/or programmer skilled in the art using the information provided herein.

Two Target Test
Frequency

```
Steps:
  1. Set MaxTime=30
  2. Set TapCounter=0
  3. Set Counter=MaxTime
  4. While Counter>0 Do
    1. Set Taps=number of taps
    2. Set TapCounter=TapCounter+Taps
    3. Set Counter=Counter−1
  5. EndWhile
  6. Set Frequency=TapCounter/MaxTime
  7. Output Frequency
```

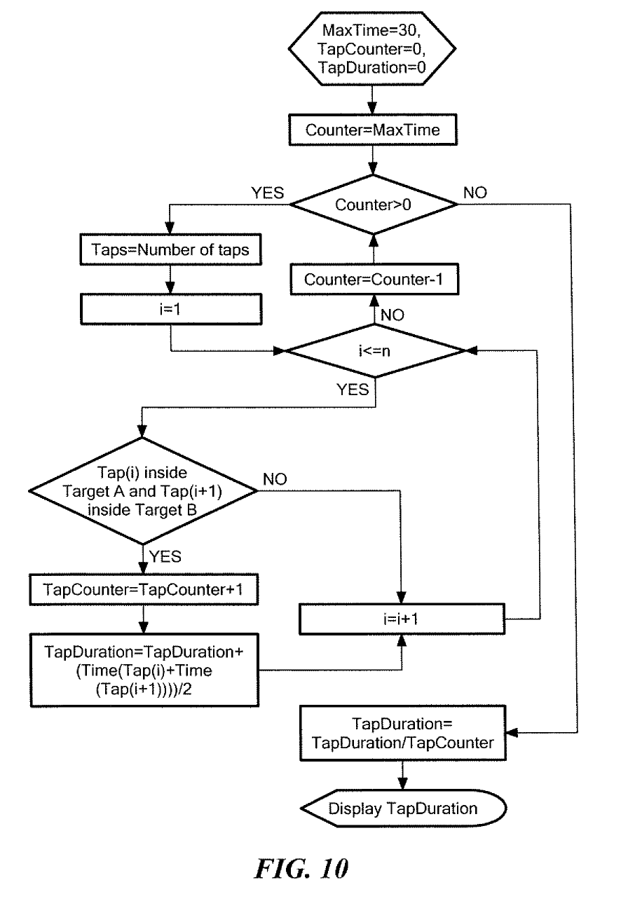

FIG. 10 is a flow chart showing the primary steps associated with one example of determining the tap duration of each tap in side or on targets 30, 32, FIG. 8 performed by tap application 16.

For enablement purposes only, the following code portion is provided which can be executed by the processor subsystem on mobile device 12 to carry out the primary steps and/or functions of the application 16 discussed above and recited in the claims hereof. Other equivalent algorithms and code can be designed by a software engineer and/or programmer skilled in the art using the information provided herein.

Duration:

```
1. Set TapDuration=0
2. Set TapCounter=0
3. Set Counter=MaxTime
4. While Counter>0 Do
  4.1. Set Taps=Number of Taps
  4.2. For i=1 to Taps Do
    a) If (Tapi inside Target A) and (Tapi+l inside Target B) Then
      Set TapDuration=TapDuration+(Time(Tapi )+Time(Tapi+1))/2
      Set TapCounter=TapCounter+1
    b) EndIf
  5.3. EndFor
  5.4. Set Counter=Counter−1
6. EndWhile
7. Set TapDuration=TapDuration/TapCounter
8. Output TapDuration
Where Time( ) is a function measuring the duration of a Tap in miliseconds.
```

Similarly, as discussed above with reference to at least FIG. 1, tap application 16, FIG. 8, is also configured to determine the accuracy of each of the taps inside or on each of spaced targets 30, 32 to further determine the motor performance of the human subject. The accuracy may include measuring the distance between the location of each tap on screen 14 and the distance from center point 34 or 36 of the intended target 30, 32 to further determine the motor performance of the human subject. Concentric rings 38, 40 of target 30 and concentric rings 42, 44 of target 32 may also be used to determine the accuracy of the taps on targets 30, 32, respectively.

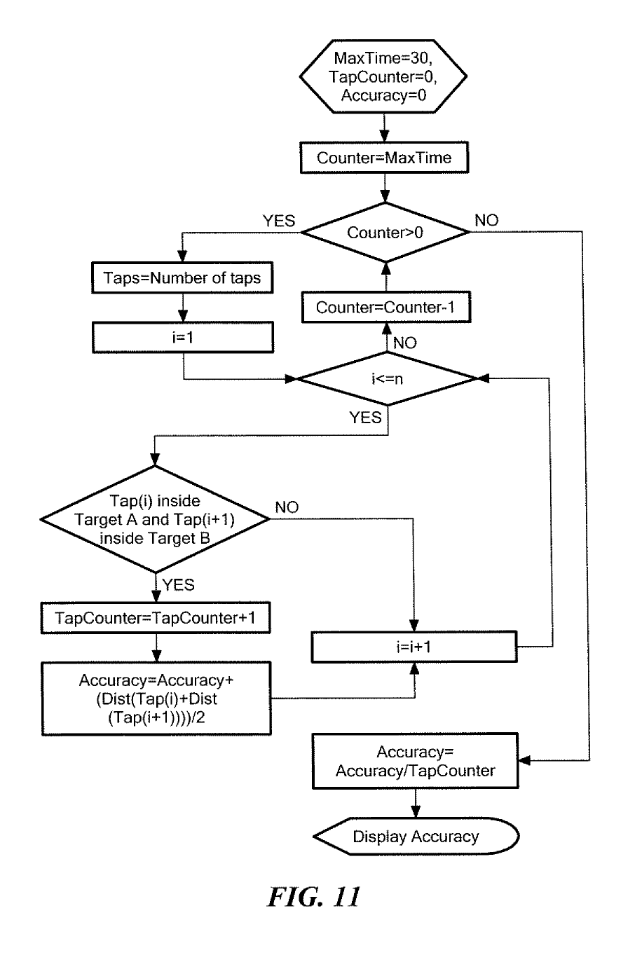

FIG. 11 is a flow chart depicting one example of the primary steps associated with calculating the accuracy of taps by the human subject inside or on targets 30, 32, FIG. 8, performed by tap application 16 to determine the motor performance of a human subject.

For enablement purposes only, the following code portion is provided which can be executed by the processor subsystem on mobile device 12 to carry out the primary steps and/or functions of the application 16 discussed above and recited in the claims hereof. Other equivalent algorithms and code can be designed by a software engineer and/or programmer skilled in the art using the information provided herein.

Accuracy—Two Target Test:

```
1. Set MaxTime=30
2. Set TapCounter=0
3. Set Accuracy=0
4. Set Counter=MaxTime
5. While Counter>0 Do
  5.1. Set Taps = Number of taps
  5.2. For i=1 to Taps Do
    a) If (Tapi inside Target A) and (Tapi+1 inside Target B) Then
      Set TapCounter=TapCounter+1
      Set Accuracy=Accuracy +(Dist(Tapi,TargetA)+
      Dist(Tapi+1,TargetB))/2
      b) EndIf
  5.3. EndFor
  5.4. Set Counter=Counter−1
6. EndWhile
7. Set Accuracy=Accuracy/TapCounter
8. Output Accuracy
Where Dist(X,Target) is a function which measures the distance from the center of Target.
```

Tap application 16, FIG. 8, may also be configured to measure the tapping velocity of alternating taps between targets 30, 32 in a predetermined pattern to further determine the motor performance of a human subject. In one example, the predetermined pattern may be simply alternating back and forth between targets 30, 32.

In another example, the predetermined pattern displayed by tap application 16 may include a complex, static or moving pattern of taps on spaced targets 30, 32 to measure the motor and cognitive static or moving performance of the human subject. In this example, tap application 16 is configured to measure the accuracy and duration of correct taps inside or on each of spaced targets 30, 32 and the tapping velocity between spaced targets 30, 32 to determine the motor and cognitive performance of the human subject. In one example, the predetermined pattern may include a complex, static or moving pattern that may include tapping targets using a tapping ratio for number of taps on three different spaced targets (e.g., two taps on target 1, one tap on target 2 and two taps on target 3) using sensory cues with or without background distracters and motivational techniques

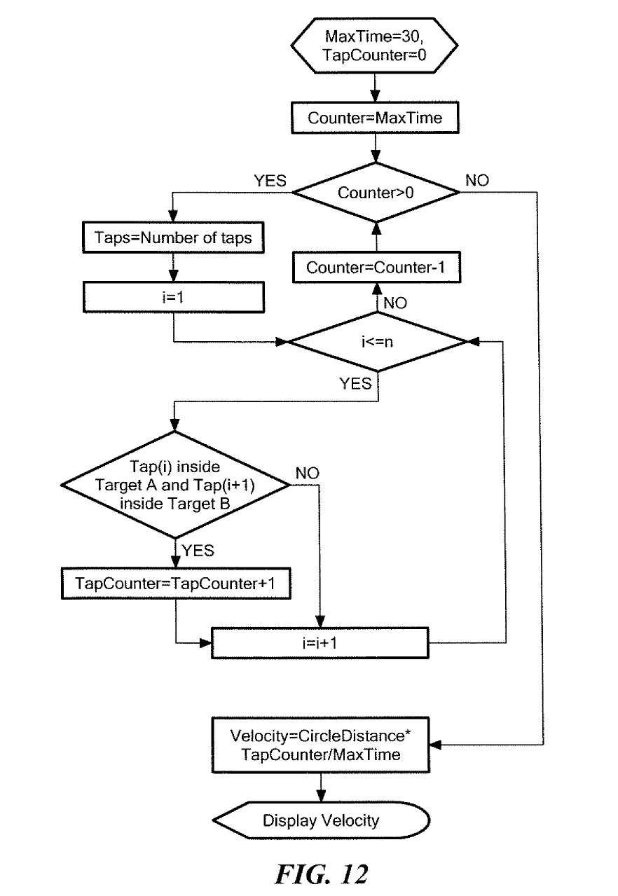

FIG. 12 is a flow chart showing one example of the primary steps associated with determining the velocity of taps between spaced targets 30, 32, FIG. 8, executed by tap application 16.

For enablement purposes only, the following code portion is provided which can be executed by the processor subsystem on mobile device 12 to carry out the primary steps and/or functions of the application 16 discussed above and recited in the claims hereof. Other equivalent algorithms and code can be designed by a software engineer and/or programmer skilled in the art using the information provided herein.

Velocity

```
6. Set MaxTime=30
7. Set TapCounter=0
8. Set Counter=MaxTime
9. While Counter>0 Do
  9.1 Set Taps = Number of taps
  9.2. For i=1 to Taps do
    a) If (Tapi inside Target A) and (Tapi+1 inside Target B) Then
      Set TapCounter=TapCounter+1
      b) EndIf
  4.3. EndFor
  4.4. Set Counter=Counter−1
5. EndWhile
6. Set Speed=TapCounter*CircleDistance/MaxTime
7. Output Speed
Where CircleDistance is the distance between the centers of two circles.
```

The result is system 10 for testing the motor and/or cognitive performance of a human subject with mobile device 12, shown in one or more of FIGS. 1-12, can measure the frequency, duration, and accuracy of taps on or inside one or more targets to accurately, efficiently, and effectively determine the motor and/or cognitive performance of the human subject. The motor and/or cognitive performance of the human subject determined by system 10 may be used by the healthcare professional and researchers to provide diagnostic values, therapeutic values, and monitor disease.

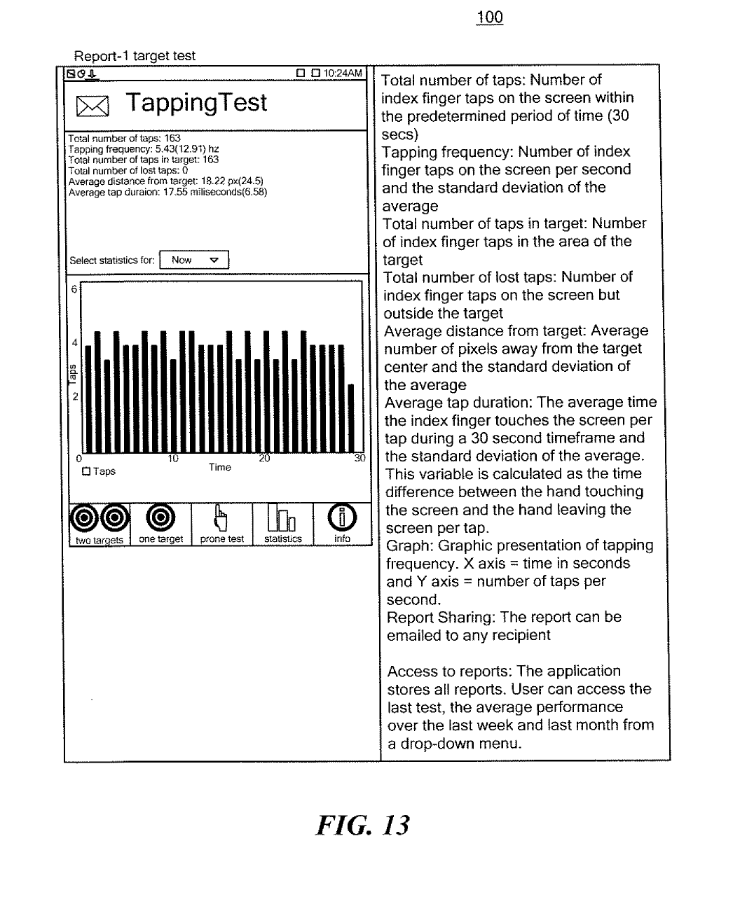

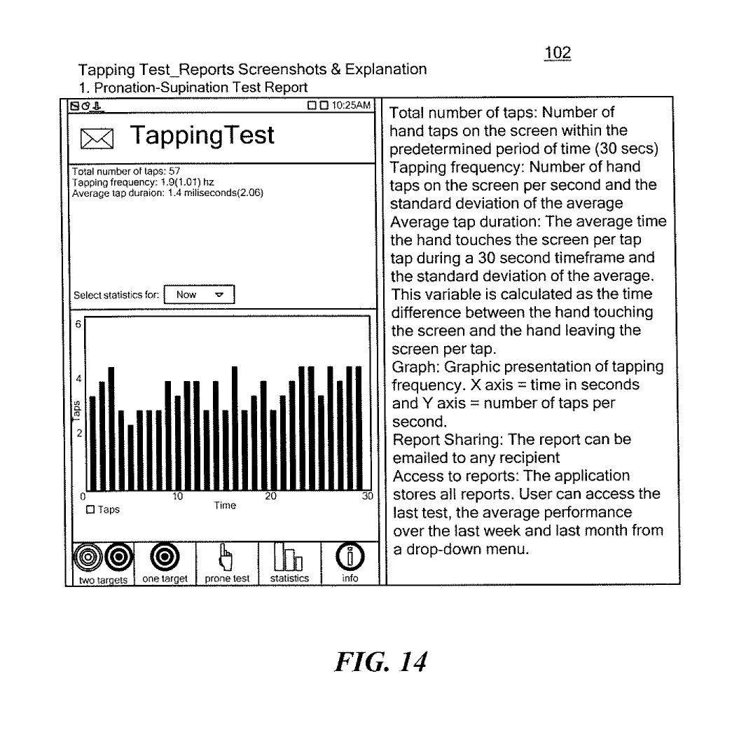

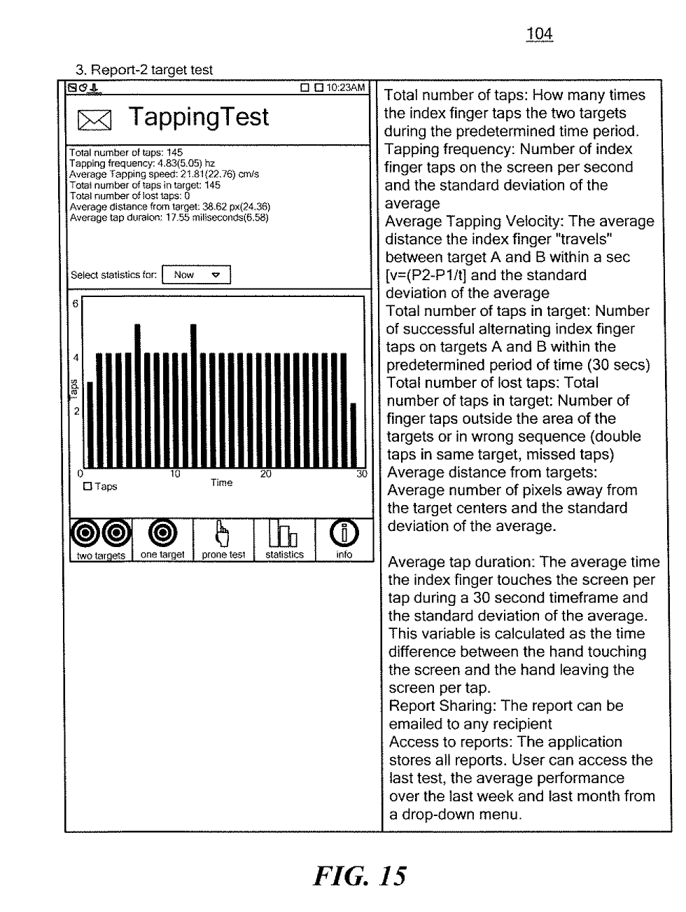

System 10 may generate a plurality of reports which may be used by healthcare professionals and researchers as complementary data for characterizing disease profiles discussed in detail in the Background section above. Exemplary reports may include target test report 100, FIG. 13, for system 10, FIG. 1, pronation-supination test report 102, FIG. 14, for system 10, FIGS. 2, and 2 target test report 104, FIG. 15 for system 10, FIG. 8. Other equivalent reports may be generated by those skilled in the art. Tap application 10, shown in one or more of FIGS. 1-15 preferably stores all the reports on mobile device 12. For enablement purposes only, the following code portions are provided which can be executed on processor subsystem 13 mobile device 12 to carry out the primary steps and/or functions, of tap application 16 shown in FIGS. 1-15 and recited in the claims hereof. Other equivalent algorithms and code can be designed by a software engineer and/or programmer skilled in the art using the information provided herein.

### App Information Page Code

In addition, any amendment presented during the prosecution of the patent application for this patent is not a disclaimer of any claim element presented in the application as filed: those skilled in the art cannot reasonably be expected to draft a claim that would literally encompass all possible equivalents, many equivalents will be unforeseeable at the time of the amendment and are beyond a fair interpretation of what is to be surrendered (if anything), the rationale underlying the amendment may bear no more than a tangential relation to many equivalents, and/or there are many other reasons the applicant cannot be expected to describe certain insubstantial substitutes for any claim element amended.

What is claimed is:

1. A system for testing motor and/or performance of a human subject with a mobile device, the system comprising:
a mobile device including a processor subsystem and configured to receive taps from a hand or part of a hand of a human subject on a screen of the mobile device; and
a tap application for the mobile device executed by the processor subsystem and responsive to taps by the hand or part of the hand on the screen and configured to measure the frequency and duration of each tap by the hand or part of the hand on the screen to determine the motor and/or cognitive performance of the human subject.

2. The system of claim 1 in which the tap application is configured to calculate the duration of each tap by determining the total amount of time the hand or part of the hand touches the screen per tap during a predetermined amount of time.

3. The system of claim 1 in which the taps by the hand or part of the hand includes tapping on the screen with a finger or thumb of the hand.

4. The system of claim 1 in which the taps by the hand or part of the hand on the screen includes alternatively tapping on the screen with the palm and dorsal surface of the hand in a repetitive pronation-supination movement.

5. The system of claim 1 in which the tap application is configured to display a target of a predetermined shape having a center point on the screen.

6. The system of claim 5 in which the tap application is configured to deter mine the accuracy of the taps on or near the center point of the target to further determine the motor performance of the human subject.

7. The system of claim 6 in which the tapping application is configured to determine the accuracy of the taps on or near the center point by measuring the distance from the location of each tap on the screen and the center point of the target.

8. The system of claim 1 in which the tap application is configured to display a plurality of spaced targets of a predetermined shape and each including a center point on the screen, the tapping application configured to measure the frequency and duration of taps inside or on each of the spaced target by the hand or part of the hand to determine the motor performance of the human subject.

9. The system of claim 8 in which the tap application is configured to calculate the average duration of each tap by determining the total amount of time the hand or part of the hand touches each of the spaced target per tap during a predetermined amount of time and dividing the total amount of time.

10. The system of claim 8 in which the tapping application is configured to measure the accuracy of the taps inside or on the spaced targets to determine the motor performance of the human subject.

11. The system of claim 10 in which the tapping application is configured to determine the accuracy by measuring the distance between the location of each tap on the screen and a center point of an intended spaced target.

12. The system of claim 8 in which the tapping application is configured to measure tapping velocity of alternating taps between the spaced targets in a predetermined pattern to determine the motor performance of the human subject.

13. The system of claim 12 in which the predetermined pattern includes alternately tapping inside or on the spaced targets.

14. The system of claim 12 in which the predetermined pattern includes a complex pattern of taps inside or on the spaced targets to measure cognitive performance of the human subject.

15. The system of claim 5 in which the predetermined shape includes circular shape having one or more concentric rings about the center point.

16. The system of claim 8 in which the predetermined shape includes circular shape having one or more concentric rings about the center point.

17. The system of claim of 1 in which the mobile device includes a mobile smart phone, a tablet device, a laptop computer, a portable personal computer, or a notebook.

18. The system of claim 1 in which the tap application is configured to generate one or more reports providing an indication of the motor and/or cognitive performance of the human subject.

19. The system of claim 17 in which the one or more reports include one or more of one a target test report, a pronation-supination report, and a two target test report.

20. A system for testing the motor and/or cognitive performance of a human subject with a mobile device, the system comprising:
a mobile device including a processor subsystem screen configured to receive taps from a hand or part of a hand of a human subject on a screen of the mobile device; and
a tapping application for the mobile device executed by the processor subsystem configured to display a plurality of spaced targets of a predetermined shape on the screen and configured to measure the frequency, duration, and accuracy of taps inside or on the spaced targets by the hand or part of the hand in a predetermined pattern to determine the motor and/or cognitive performance of the human subject.

21. A system for testing the motor and cognitive performance of a human subject, the system comprising:
a mobile device including a processor subsystem and configured to receive taps by a hand or part of the hand of a human subject on a screen of the mobile device; and
a tap application for the mobile device executed by the processor subsystem configured to display a plurality of targets in a predetermined complex static or moving pattern on the screen and configured to measure the accuracy and duration of correct taps inside or on each of the plurality of spaced targets and the tapping velocity of taps between the spaced targets to determine the motor and cognitive performance of the human subject.

22. A method for testing the motor and/or cognitive performance of a human subject with a mobile device, the method comprising:
receiving taps from a hand or part of a hand of a humans subject on a screen of the mobile device; and
measuring the frequency and duration of each tap by the hand or part of the hand on the screen to determine the motor performance of the human subject.

23. The method of claim 22 further including calculating the duration of each tap by determining the total amount of time the hand or part of the hand touches the screen per tap during a predetermine amount of time and dividing the total amount of time by a total number of taps during the predetermined amount of time.

24. The method of claim 22 including the step of displaying a target of a predetermined shape and having a center point on the screen.

25. The method of claim 24 further including determining the accuracy of taps on or near the center point of the target to further determine the motor performance of the human subject.

26. The method of claim 25 in which the accuracy includes measuring the distance from the location of each tap on the screen and the center point of the target.

27. The method of claim 22 further including displaying a plurality of spaced targets of a predetermined shape and each having a center point on the screen and measuring the frequency and duration of taps inside or on each of the spaced targets to determine the motor performance of the human subject.

28. The method of claim 27 further including measuring the accuracy of taps inside or on the spaced targets to determine the motor performance of the human subject.

29. The method of claim 27 further including measuring the tapping velocity of alternating taps between the spaced targets in a predetermined pattern to determine the motor performance of the human subject.

30. The method of claim 29 in which the predetermined pattern includes alternatingly tapping inside or on the spaced targets.

31. A method for testing the motor and/or cognitive performance of a human subject with a mobile device, the method comprising:
receiving taps from a hand or part of a hand of a human subject on a screen of the mobile device; and
displaying a plurality of spaced targets of a predetermined shape on the screen and measuring the frequency, duration, and accuracy of taps inside or on the spaced targets by the hand or part of the hand in a predetermined pattern to determine the motor and/or cognitive performance of a human subject.

32. A method for testing the motor and/or cognitive performance of human subject with a mobile device the method comprising:
receiving taps by hand or part of the hand of a human subject on a screen of a mobile device; and
displaying a plurality of targets in a predetermined complex static or moving pattern on the screen and measuring the accuracy and duration of correct taps inside or on each of the plurality of spaced targets and determining the tapping velocity of taps between the spaced targets to determine the motor and cognitive performance of a human subject.
